(19) United States
(12) Patent Application Publication
(10) Pub. No.: US 2025/0154546 A1
(43) Pub. Date: May 15, 2025

(54) USE OF 3'-OXYMETHYLENE ALKYL DISULFIDE PROTECTED NUCLEOTIDES FOR ENZYMATIC DNA AND RNA SYNTHESIS

(71) Applicant: Miltenyi Biotec B.V. & Co. KG, Bergisch Gladbach (DE)

(72) Inventors: Sano Mong Marma, Bergisch Gladbach (DE); Sameh Soliman, Bergisch Gladbach (DE); Xiao-Pei Guan, Bergisch Gladbach (DE); Michel Perbost, Bergisch Gladbach (DE); Robert Pinard, Lowell, MA (US)

(21) Appl. No.: 18/837,250

(22) PCT Filed: Feb. 10, 2023
(86) PCT No.: PCT/EP2023/053268

§ 371 Date: Aug. 9, 2024

(30) Foreign Application Priority Data
Feb. 11, 2022 (EP) 22156251.5

(51) Int. Cl. C12P 19/34

(52) U.S. Cl. CPC C12P 19/34

(57) ABSTRACT

This invention is about enzymatic synthesis of nucleic acids using 3′-O—(CH₂SSR) protected reversible nucleotide terminators. The nucleotide base of the protected nucleotide may consist of natural or non-natural (e.g. 7-de-aza G and 7-de-aza A), or mixture thereof. The base of the nucleotides can be modified with a linker carrying carboxylic acid (—CO₂H). amine (—NH₂), thiol (—SH). hydroxymethyl (—CH₂OH), propargy lamine. alkyne group etc. for subsequent modification and labeling. Such nucleotide substrates can be incorporated into the single strand, template free nucleic acid or into the templated DNA hybrid. And in later step the 3′-O—CH₂SSR protecting group can be cleaved off by chemical treatment pre-requisites for enzymatic nucleic acids synthesis. By adding nucleotide in pre-determined fashion and cleave reaction after each step. longer DNA strands can be synthesized in solution or on solid surface starting from a short seeding DNA strands.

### BACKGROUND

The technologies for DNA sequencing and the DNA synthesis are very fundamental to modern biology. The DNA sequencing methods allow reading the genetic code while the DNA synthesis methods allow laboratory manufacture of the genes and create their products.

Between them, the latter method significantly lags behind in many aspects. Massively parallel method of DNA sequencing has been outpacing the DNA synthesis method, more significantly since the past two decades due to introduction of new-generation DNA sequencing technologies (NGS technologies).

On the other hand, the DNA synthesis method has not changed fundamentally in the past couple decades. DNA synthesis is required in many branches in biology from developing diagnostic kits to producing active pharmaceutical ingredients (API) for nucleic acid therapies to constructing DNA based data storage systems. Currently nucleic acids synthesis are mainly done on solid support using phosphoramidite chemistry-developed by Marvin Caruthers and colleagues at the University of Colorado in 1981.

This phosphoramidite method is not environmental friendly as reactions take place in organic phases and produces significant amount of organic wastes. Further, this method can produce only short stretches of nucleic acids reaching to its limitation at about 200 nucleotides. On the other hand, the most gene constructs require in the ranges from 2,000 to 3,000 nucleotides. For longer length oligonucleotides, only one viable way so far is PCR reactions using shorter synthetic nucleic acids with overlapping segments for stitching together and amplifying step by step to generate longer stretches of DNA molecules. Often such method is time consuming and cost prohibitive.

To overcome this limitation, there has been considerable effort in developing alternative method for DNA synthesis. The main reasons are to achieve the synthesis of longer strand of DNA molecules rapidly, accurately and efficiently, also be able to create many DNA sequences simultaneously, environmental friendliness and overall cost reduction.

One of the methods that has been drawing significant interest in this regard is template free DNA synthesis using reversibly terminating nucleotides and TdT enzyme. In this method, the nucleotide is added once at a time-terminate by single extension followed by cleavage. This requires nucleotide with capping group on 3′-OH (WO 2016/128731 A1. US 10,774,316 B2). The other method, which works similarly, involves the use of nucleotide directly linked to TdT via a cleavable linker (Nature Biotechnology, vol 36, pages 645-650,2018). In both cases, the first step is adding nucleotides in predetermined fashion to stop DNA synthesis by single base incorporation. Then regenerating the n+1 strain by chemical treatment for making the growing strand available for the incorporation of the subsequent bases.

This is to great extent similar to how the most powerful DNA sequencing method, often known as sequencing by synthesis (SBS) works. In both cases, the 3′-OH group of the nucleotide must be capped with a smaller functional group for enzymatic incorporation. And more importantly, the capping group must be removed by chemical treatment under a condition congenial to fragile DNA molecules. Therefore, reversible nucleotide terminators that are proven to be useful in sequencing by synthesis is also likely to meet the two basic requirements for enzymatic DNA synthesis-the terminating by single base and cleavability under DNA compatible condition.

3′-OH protected nucleotides, especially, 3′-O-CH₂SSMe are known to be accepted by polymerase to terminate DNA synthesis by single base incorporation due to smaller size of it. In subsequent step, it can be removed using benign chemicals such as with thiols and phosphines. Such technology is disclosed in U.S. Pat. Nos. 10,301,346, 10,273,539 and 10,336,785 in use with a template oligonucleotide in sequencing process.

Many nucleotide classes are good substrate for enzymatic incorporation into growing strand of DNA molecules but impossible to cleave under benign, DNA compatible conditions limiting their uses in DNA sequencing. Such nucleotides cannot be an option in direct DNA and RNA synthesis i.e. without the use of a template strand either because cleavability under DNA and RNA friendly condition is the essential aspect in both the DNA sequencing and the synthesis method.

Accordingly, it was the objective of the present invention to provide blocked nucleotides for direct DNA and RNA synthesis by enzymatic reaction.

### SUMMARY

It was found that 3′-OH protected nucleotides, especially, 3′-O—CH₂SSMe known from sequencing-by-synthesis processes can be used in direct, template free, enzymatic synthesis of DNA and RNA synthesis.

Object of the invention is therefore a method for synthesis of DNA or RNA strands comprising the steps

a) Providing a primer capable of binding to nucleotides
b) Providing a first nucleotide according to general formula (I)

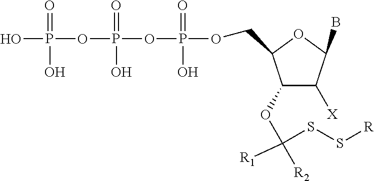

With
B=natural or non-natural nucleobase
R=—Me, —Et, —Pr, —iPr, —iBu, CH₂CH₂NH₂, CH₂CH₂CN
R1, R2=H, D, —Me, —Et, —Pr, —iPr, —iBu, CHF₂CH₂CH₂F
   c) providing terminal transferase enzyme thereby ligating the first nucleotide to the primer
   d) cleaving and removing the protection group SS-R from the first nucleotide
   e) repeating steps b) to d) by providing further nucleotides according to general formula (I) thereby obtaining DNA or RNA strands

The method of the invention is not directed to obtain sequencing information of the DNA or RNA strands, but to a method for synthesis for DNA or RNA strands. Therefore, in a preferred embodiment the first and further nucleotides provided in step b) and e) according to general formula (I) do not comprise a detectable label. The term “detectable label” covers all labels used in the technology of sequencing by synthesis like dyes, especially fluorescent dyes, mass tags and radioactive tags.

In theory, steps b) to d) (i.e. step e)) can be repeated as often as necessary to obtain DNA or RNA strands having the desired length. However, in praxis steps b) to d) (i.e. step e)) are usually repeated 10 to 1000 times.

Nucleotides with 3′-OCH₂SSMe group terminate DNA synthesis by single base extension and it can be further extended at will when treated with TCEP or thiol compounds. TCEP and Thiol compounds are mild and work well under DNA compatible conditions, which also make them excellent candidates for DNA and RNA synthesis.

With the method of the invention, enzymatic synthesis of nucleic acids using 3′-O—(CH2SSR) protected reversible nucleotide terminators is possible. The nucleotide base of the protected nucleotide may consist of natural or non-natural (e.g. 7-de-aza G and 7-de-aza A), or mixture thereof. The base of the nucleotides can be modified with a linker carrying carboxylic acid (—CO2H), amine (—NH2), thiol (—SH), hydroxymethyl (—CH2OH), propargylamine, alkyne group etc. for subsequent modification and labeling. Such nucleotide substrates can be incorporated into the single strand, template free nucleic acid or into the templated DNA hybrid. And in later step the 3′-O—CH2SSR protecting group can be cleaved off by chemical treatment pre-requisites for enzymatic nucleic acids synthesis. By adding nucleotide in pre-determined fashion and cleave reaction after each step, longer DNA strands can be synthesized in solution or on solid surface starting from short seeding DNA strands.

### BRIEF DESCRIPTION OF THE DRAWINGS

FIG. 1 shows nucleotide analogues with 3′OH group capped with —CH2SSR. The base (B) can be natural and non-natural nucleobase. R group can be —Me, —Et etc. The substituent X can —H, —OH, or —OH group capped with —Me, —Et etc.

FIG. 2 shows preferred nucleotide structure where the capping group is —CH2SSMe.

FIG. 3 shows chemical structures some of the cleave reagents, such as DTT, DMPS, TCEP and THPP.

FIG. 4 shows the cleavage mechanism of the 3′-O-CH2SSMe protected nucleotides by phosphine or thiol compounds. It involves two step process. First step can be instantaneous reaction while second is slow, rate limiting step. Mg2+ can speed up the second step, hydrolysis reaction.

FIG. 5 show a data plot to obtain the reaction constant

FIG. 6 shows the half-life of various cleavage reactions

### DETAILED DESCRIPTION

This invention is about the use 3′-O-methylene alkyl disulfide blocked nucleotides (3′-OCH₂SSR) in the enzymatic synthesis of DNA and RNA molecules. The primary aspect of this invention is that such nucleotides, can be built with varying sizes depending on the structure of the R group. Among them the smallest possible size is when R=Me. It is accepted by polymerase enzymes used in DNA sequencing. Because of small size it fits in the active site of the polymerase and has been demonstrated to be showing fast kinetics. The important aspect is that the capping group is stable, yet it can be easily removed under very mild DNA compatible conditions and at fast chemical reaction in about 1.0 minute.

The current invention is based on compounds of the general structure (I) which have been proven to be excellent substrate for enzymatic incorporation when engineered polymerase is used, such as terminator 3, T9 etc. And most importantly the capping (3′-OCH2SSR) can be removed gently without damaging DNA molecules.

In one aspect the nucleobases can be natural and their modification (A, T, G, C, inosine, 5′-hydroxymethyl C and T etc) or non-natural nucleotides such as 7-de-aza-G and 7-de-aza-A and their modification, the base B=non-natural base or base modified.

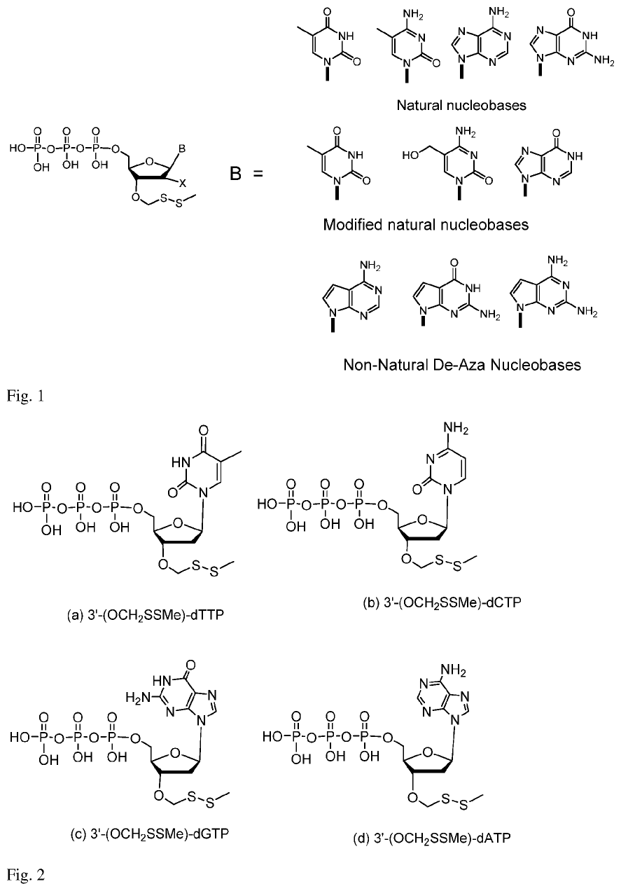

Preferable, the 3′-OH capping is methylene alkyl disulfide (3′-O—CH₂SSR) where is R group is methyl (R=-Me). FIGS. 1 and 2 show preferred nucleotides according to general formula (I).

X in formula (I) may be selected from the group consisting of hydrogen, hydroxyl, halogens, —OMe, —OEt, —OCH₂SSMe, —OCH₂CH₂NH₂.

The primer can be a nucleic acid initiator or seeding strand like a DNA, RNA or other polynucleotide strand. They can be single strand or double strand. Preferable, such DNA or RNA strand can have a length of 1 to 50 nucleotides.

In another embodiment of the invention, the primer can be bound to a solid surface like polymer surface as known for microfluidics systems, to a particle or a microsphere or nanosphere bead. Preferable, the primer is a DNA or RNA strand bound to such a solid surface.

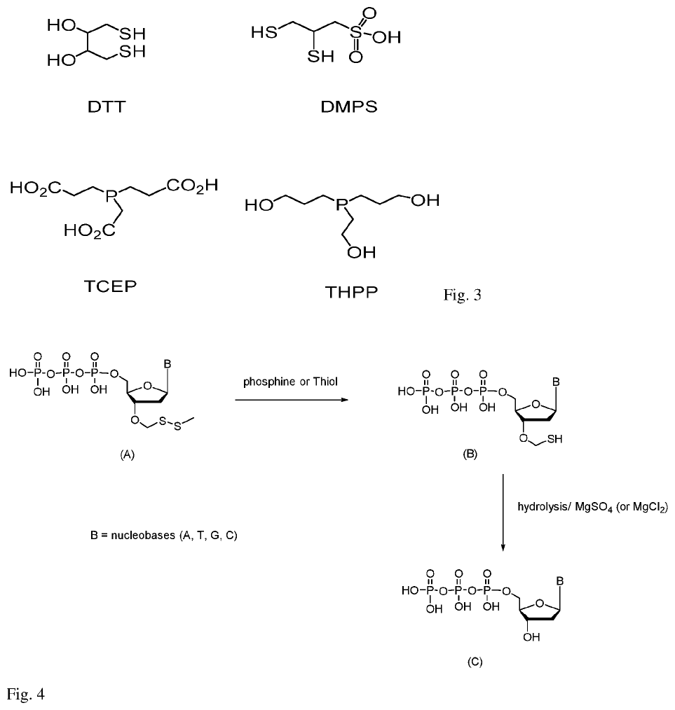

In another variant, the method may involve a chemical treatment to remove the 3′-OH capping group by a cleave reagent. Such cleave reagent may be selected from the group consisting of thiols-for example dithiolthreitol (DTT), dimercapto propanesulfonic acid (DMPS), etc. or phosphines such as tris (2-carboxyethyl) phosphine (TCEP), tris(3-hydroxypropyl) phosphine (THPP). Preferred cleave reagents are shown in FIG. 3.

Further, cleaving may be performed by adding divalent ions such as Mg2+. In praxis, salts such as MgSO₄ or MgCl₂ are added. Preferable, divalent ions are added in a pH range 7-10 for accelerating the cleave step in DNA and RNA synthesis in solution or solid phase. Most preferred is the addition of dimercapto propanesulfonic acid (DMPS) or other thiol compound or phosphine as cleave reagent added with or without Mg2+salt. FIG. shows this embodiment.

### EXAMPLES

The general workflow to obtain compounds according to formula (I) is shown in the following scheme 1:

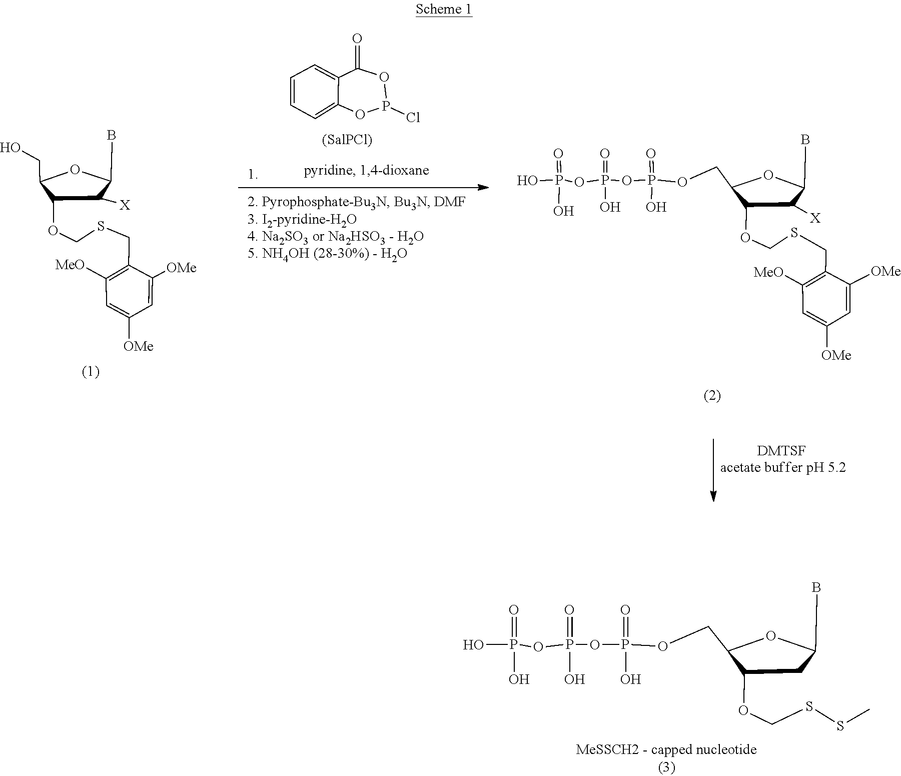

The 3′-O-CH₂SSMe capped nucleotide synthesis starts with 3′-OCH₂-S-trimethoxybenzyl analogue (compound 1). It is triphosphorylated by standard method such as Ludwig or Eckstein method and after removing all the protecting groups of the base by NH₄OH treatment, the volatile compounds and solvents are removed by lyophilization (or by high vacuum pump). It is then re-suspended in water and treat the crude product with DMTSF in acetate buffer in pH 4-6. The reaction is complete in 10 minutes. After neutralization with triethylamine to adjust pH 8.5, the crude can be stored or immediately purified by ion-exchange chromatography resulting the desired product (compound 3) in ˜25-40% yield. This method allows production of the MeSSCH₂-capped reversible nucleotide terminators (MeSSdNTPs) in higher yield, and fully scalable to higher scale by linearly increasing reagents and solvent using standard laboratory equipment quickly cutting the cost of nucleotide production significantly.

The cleave off process takes place in two steps—the first step is reductive cleavage of the disulfide bond and then hydrolysis of the resultant thiol compound as shown in FIG. 4. It was found that the first step-reductive cleavage is instantaneous, and the second step is slower and rate limiting step. The second step be accelerated by adding salt (e.g. MgSO₄, MgCl₂).

### Synthesis of 3′-O—(MeSSCH₂)-dGTP (6)

The synthesis of 3′-O—CH₂SSMe capped G nucleotide is shown in the following scheme 2:

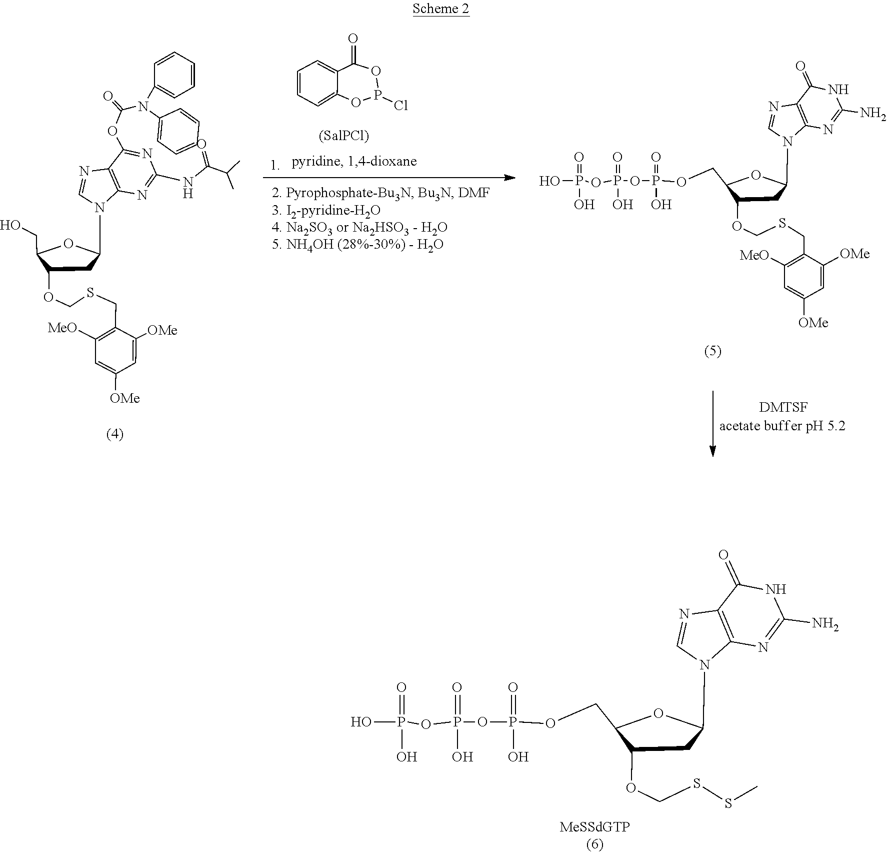

In a 250 mL flask, 1.896 g (2.50 mmole) of 5′-OH-3′-O—(TMPMT)-dG (DPC)-Bu (4) was dried overnight under high vacuum in a desiccator in the presence of P₂O₅. It was dissolved by adding 4.0 mL dry pyridine and 7.0 mL 1,4-dioxane. The mixture was treated with 2-chloro-1,3,2-benzodioxaphosphorin-4-one (0.6 g, 2.96 mmole). The mixture was stirred at room temperature for 15 mins. A solution of tributylammonium pyrophosphate (1.8 g) was prepared by dissolving in 5.0 mL dry DMF and 1.8 mL Bu₃N in a 50.0 mL centrifuge tube. The mixture was added to the vigorously stirred solution at once and resulting mixture was stirred for 10 minutes at room temperature. To the reaction mixture, I₂ (0.72 g. 2.84 mmole) solution, prepared in 5.0 mL pyridine and 0.5 mL HPLC grade water was added. The resulting mixture was stirred for 15 minutes. The excess iodine was then quenched by adding 1.0 mL of 5% Na₂SO₃ in HPLC grade water. After stirring for 5 minutes, the reaction was diluted with 80 mL HPLC grade water. The mixture was stirred for 1.5 hour at room temperature. The resultant solution was transferred to a 1,000 mL round bottom flask by decantation leaving behind the sticky-reddish precipitates. The mixture was treated with 240 mL of 28-30% NH₄OH, stirred for 24 hours at room temperature. The mixture was concentrated to dryness by lyophilization. The mixture was re-suspended in HPLC grade water and filtered to result in 120 mL crude filtrate solution of compound 5. The resultant crude product was diluted with 12 mL 3.0 M acetate buffer at pH 5.2. To rigorously stirring solution of this, 1.6 gm of dimethyl (methylthio) sulfonium fluoroborate (DMTSF, 8.2 mmole) was added and the mixture was stirred at room temperature for ˜10 minutes. The resultant turbid mixture was neutralized with ˜8.0 mL Et₃N and precipitates were removed by centrifuge. The final product was obtained after ion-exchange purification on PL-SAX column (10 μ, 1,000A, 50×150 MM, Agilent, method: 0-5 min 100% A, followed by 80% B over 50 min, flow rate −40 mL/min; A=15% ACN/H₂O, B=15% ACN/0.85 M TEAB buffer) followed by de-salting by flash chromatography on 275 g C18Aq Teledyne column resulted in 691 μmoles compound 6 (27.6% yield) after lyophilization and re-suspension in HPLC grade water (method: flow rate 80 mL/min, A=100 mM TEAB, B=ACN, equilibration 15 min in 100% A, gradient method: 0-5 min 100% A, followed by 25% B over 5-45 min).

### Synthesis of 3′-O—(MeSSCH₂)-dCTP (9):

The synthesis of 3′-O—CH₂SSMe capped C is shown in the following scheme 3:

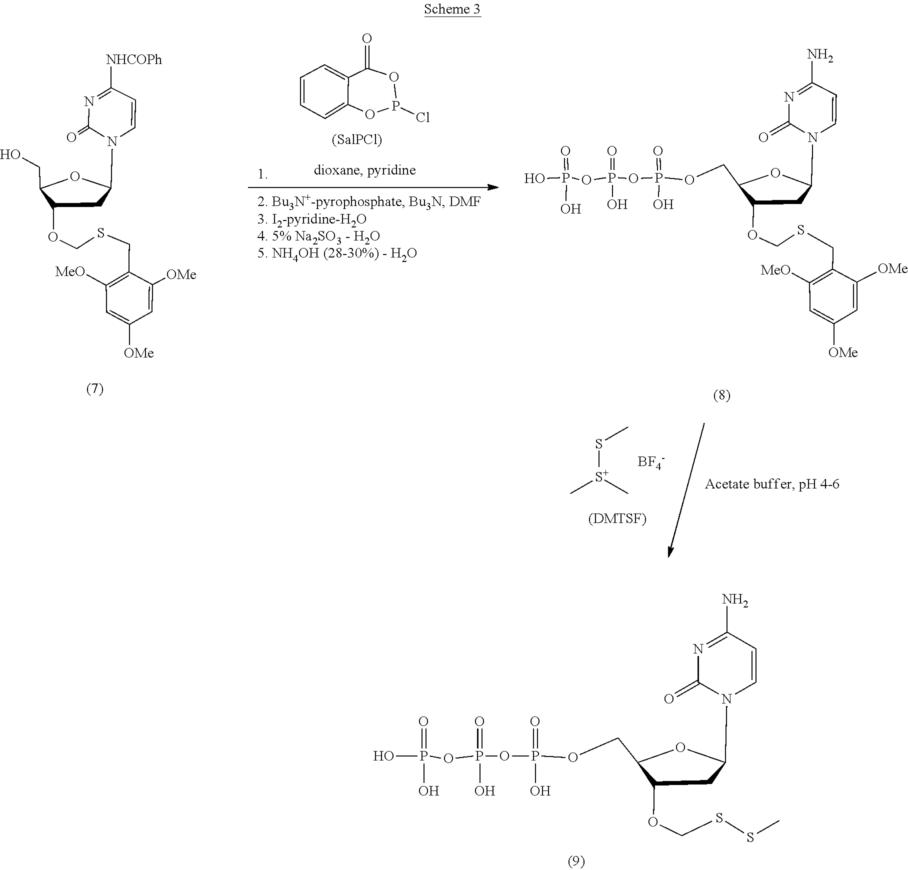

5′-OH—TMPMT-dC (Bz) 7 (3.1 g, 5.6 mmole) was transferred into a 250 ml round bottom flask. This flask, together with a magnetic stir bar and a rubber septum, were dried overnight under high vacuum in a desiccator in the presence of P₂O₅. It was dissolved in anhydrous pyridine (8.4 mL) and anhydrous 1,4-dioxane (16.8 mL), and was added partially crushed SalPCl solid (1.26 g, 6.2 mmole). The reaction mixture was stirred at room temperature for 30 minutes. A solution of tributylammonium pyrophosphate (3.8 g) in a mixture of Bu₃N (3.9 mL) and anhydrous DMF (11.2 mL) was prepared in a 50 mL centrifuge tube. It was then added at once to the reaction mixture. The resulting reaction mixture was stirred at room temperature for 15 minutes. The product was oxidized by a solution of I₂ (1.56 g, 6.2 mmole) in pyridine (11.2 mL) and H₂O (1.1 mL, HPLC grade). The resulting brown mixture was stirred at room temperature for 20 min. The excess I₂ was then quenched with 5% Na₂SO₃ (aq.) (2.3 mL). The mixture was stirred at room temperature for 5 min. The mixture was further diluted with H₂O (147 mL). It was stirred at room temperature for another 1.5 hours at room temperature. The reaction mixture was then treated with NH₄-OH (28˜30%, 110 mL). It was stirred at room temperature for 2 hours and complete de-protection was confirmed by HPLC and LCMS. The reaction mixture was lyophilized to dryness and the residue was re-suspended by HPLC grade water and filtered using a Corning filter system (0.22 μm). It was then transferred into a 250 mL bottle to give a crude TMPMT dCTP (8) solution in H₂O (˜100 mL). The solution of TMPMT-dCTP (8) in a 250 ml. bottle was further diluted with H₂O (90 ml.), followed by the addition of acetate buffer (3.0 M, pH 5.2, 20 mL). To this mixture was added DMTSF solid (2.9 g) immediately. The resulting reaction mixture was stirred at room temperature for 20 minutes. The complete conversion was confirmed by HPLC. The reaction mixture was neutralized with triethylamine (8.4 mL) to pH 7.5 and then filtered using a Corning™ disposable bottle-top filter system (250 mL, CA 0.45 μm).

The reaction mixture was purified by ion-exchange HPLC on PL-SAX. 10 μm. 1000 Å. 50×150 mm (Agilent); Method: 0-2 min 100% A, then 80% B over 50 mins, flow rate 40 mL/min, A=15% ACN/H₂O, B=15% ACN/0.85M TEAB buffer). The target peak was collected and pooled together and lyophilized. The residue was dissolved in H₂O (HPLC) & combined to give a crude product 9 solution ˜2.4 mmol) in ˜89% purity by HPLC. The crude product was de-salted by flash chromatography (Interchim PuriFlash-450; 275 g RediSep Rf Gold HP C18 Aq column; Method: 0-10 min 100% A, then 10% B over 30 mins; A=100 mM TEAB. B=ACN; flow rate 100 mL/min). The target fractions were combined and lyophilized. The product was dissolved in 1X TE buffer and combined to give the desired pure MeSSdCTP (6) in 1X TE. Total 90 mL (1.812 μmol. 36% from 9) in >99% purity by HPLC.

### Synthesis of 3′-O—(MeSSCH₂)-dTTP (12)

The synthesis of 3′-O—CH2SSMe capped T is shown in the following scheme 4

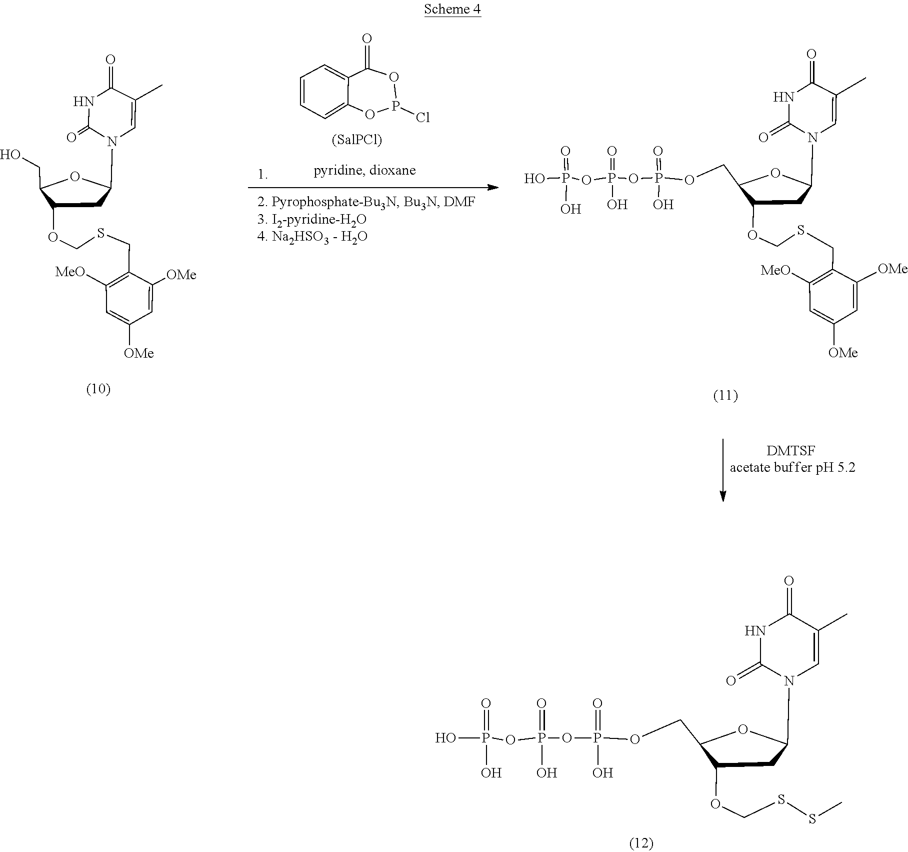

In 250 mL round bottomed flask, 4.7 g (10.06 mmole) of 5′-OH-3′-(TMPMT)-dT (10) was dried overnight under high vacuum in the presence of P₂O₅ as a drying agent. It was dissolved in 15.0 mL dry pyridine and 30.0 mL dry 1,4-dioxane under nitrogen atmosphere. The mixture was treated with 2-chloro-1,3,2-benzodioxaphosphorin-4-one (2.294 g, 11.2 mmole) under nitrogen atmosphere. The mixture was stirred at room temperature for 30 mins. Then, a solution of tributylammonium pyrophosphate (7.0 g) in 20.0 mL dry DMF and 7.0 mL Bu₃N was prepared in a 50.0 mL centrifuge tube. The mixture was added at once and stirred for 10 minutes at room temperature. The reaction product was oxidized by a solution of I₂ (2.9 g, 11.4 mmole) in 20.0 mL pyridine and 2.0 mL HPLC grade water. The mixture was stirred for 15 minutes, turning into a reddish solution. The excess iodine was then quenched by adding 4.0 mL 5% Na₂SO₃ in HPLC grade water and stirred for 5 minutes. The reaction mixture was transferred into 1000 mL flask and diluted with 300 mL of HPLC grade water. The clear solution was transferred to another new 1000 mL by decantation leaving behind the red-brown precipitates. The mixture was frozen initially on acetonitrile-dry ice bath (−40° C.) and later about 1.0 hour at −75° C. freezer, and lyophilized, which resulted in thick paste product. The product was re-suspended/extracted with HPLC grade water and the precipitates were removed by filtration and the total volume at this point is ˜480 mL (crude stock ˜21 mM 3′-(TMPMT)-dTTP, 8, based on starting material 7).

About half amount (˜240 mL, ˜5.0 mmole) of the 480 mL 3′-(TMPMT)-dTTP crude (11) was taken in a different plastic bottle. It was diluted with ˜30 mL 3.0 M acetate buffer pH 5.2. It was treated with 3.0 g of DMTSF and the mixture was stirred at room temperature for ˜20 minutes. The HPLC of the mixture showed full conversion to MeSSdTTP. It's clear mixture (slight yellowish) was taken up into another flask after removing the precipitate by centrifugation (using 50 mL falcon tube). The mixture was adjusted to pH 7.0 by neutralizing with neat Et₃N (˜15 mL). At this moment, total volume ˜300 mL was split into two, each 150 mL (2.5 mmole). It was then purified by ion-exchange HPLC on PL-SAX (10 μ, 1,000A, 50×150 MM, Agilent), gradient: 0-5 min 100% A, followed by 80% B over 5-50 min, flow: 40 mL/min, A=15% ACN/H₂O, B=15% ACN/0.85 M TEAB buffer, target peak with R_{f}=23-28 mins. The collected fractions were combined and lyophilized. The dried sample was re-suspended in 100 mM TEAB buffer and de-salted or final purified on C18 Sunfire column. Yield: 780 μmole, 31%, semi-pure product. Final purification/de-salting was carried on C18 Sunfire Waters Column (50×250 MM): Method: 0-2 min 100% A, then 40% B over mins, flow rate-40mL/min, A=100 mM, B=ACN. Yield 607 μmoles of compound 12 (˜ 24% over all yield)

### Synthesis of 3′-O—(MeSSCH₂)-dATP (15);

The synthesis of 3′-O—CH₂SSMe capped A is shown in the following scheme 5:

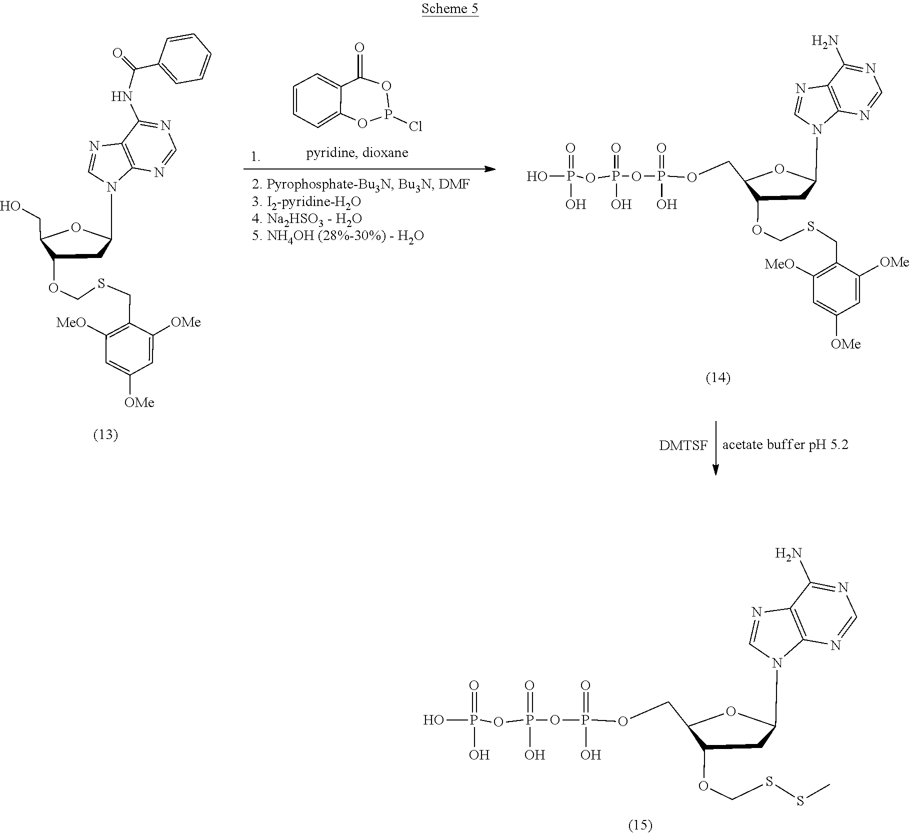

In 250 mL pears-shaped flask, 1.454 g (2.50 mmole) of 5′-OH-3′-(TMPMT)-dA-Bz (13) was dried overnight under high vacuum in the presence of P₂O₅. It was dissolved by adding 4.0 mL anhydrous pyridine and 7.0 mL anhydrous 1,4-dioxane under nitrogen atmosphere. The mixture was treated with 2-chloro-1,3,2-benzodioxaphosphorin-4-one (0.6 g, 2.96 mmole) and the mixture was stirred at room temperature for 15 mins. A solution of Bu₃NH⁺-pyrophosphate (1.8 g) in 5.0 mL dry DMF and 1.8 mL Bu₃N was prepared in a 50.0 mL centrifuge tube. The mixture was added at once and stirred for 10 minutes at room temperature. The product was then oxidized by adding a solution of I₂ (0.72 g, 2.84 mmole) prepared by dissolving in 5.0 mL pyridine and 0.5 mL HPLC grade water. The mixture was stirred for 15 minutes, turning into a reddish solution. The excess iodine was quenched by adding 1.0 mL of 5% Na₂SO₃ in HPLC grade water and stirred for 5 minutes. The mixture was diluted with 80 mL of HPLC grade water and was stirred for 1.5 hour at room temperature. The color of the mixture changed to a clear solution with red-sticky precipitations on the wall of the reaction flask. The clear solution was transferred to new 1,000 mL round bottom flask by decantation leaving behind the red precipitates. The mixture was treated with 240 mL of 28-30% NH₄OH for 8 hours. The product (14) was then lyophilized and the product was dissolved in HPLC grade water, resulting solution in ˜100 mL (˜25 mM apparent concentration, based on starting material amount). It was diluted with 12 mL 3.0 M acetate buffer pH 5.2 and treated with 1.6 g of DMTSF for 20 minutes. The resultant turbid mixture was neutralized with 6.0 mL Et₃N, precipitates were removed by centrifuge, the resulting pH˜9.0.

The product was purified by ion-exchange HPLC on PL-SAX column (10u. 1,000 A, 50×150 MM, Agilent), HPLC gradient: 0-1 min 100% A, followed by 80% B over 50 min, flow rate: 40 mL/min, A=15% ACN/H2O, B=15% ACN/0.85M TEAB buffer. Peak with Rf=25-30mins was processed for lyophilization. After lyophilization, the product was further purified on 270 gm C18AQ column (Teledyne) by single injection, flow rate 80 mL/min, A=100 mM TEAB, B=ACN, equilibration 15 min in 100% A, 0-5 min 100% A, then 25% B 5-40 min. All fraction was pulled together and lyophilized. The product MeSSdATP (15) was dissolved in 1X TE and determined concentration by UV-spectrometry to yield total volume=60 mL, C=11.88 mM, yield=712 μmoles (28.5% yield) in >98% purity.

### Example for Cleavage Reaction

### General Cleave Kinetics Methodology

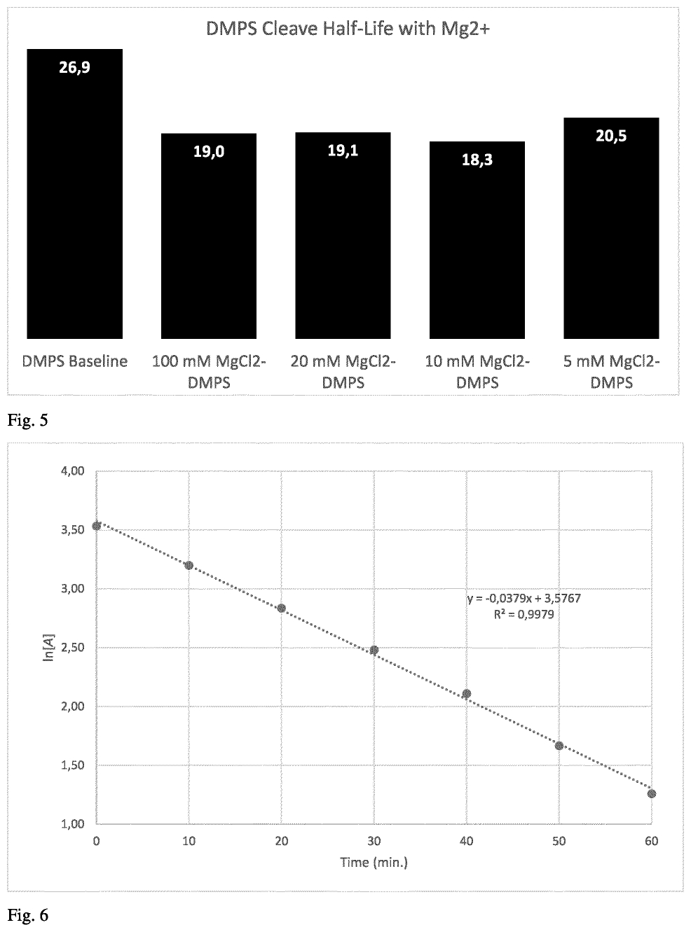

A 3′-O-capped nucleotide was treated with a cleave mix DMPS/THPP and the concentration of the 3′-capped nucleotide was monitored by UPLC with a UV detector at a fixed time interval. From these data, the reaction rate coefficient k and reaction half-life t½can be obtained as shown in FIG. 5 following the equations

In [A]=−kt+In[A]₀ and t_{1/2}=In2/k with [A]: reactant concentration; k: reaction rate coefficient

### Experimental

Nucleotide: MeSSdATP, 4.7 mM in 1× TE buffer;

Cleave mix: 0, 5, 10, 20, 50, or 100 mM MgC12 and 20 mM DMPS in buffer at pH 9.5.

12.8 μL of nucleotide was added to 287 μL of cleave mix in a 1.5 mL of UPLC sample vial. The resulting mixture was analyzed by UPLC right away and every 10 min afterwards automatically for up to 120 min. The area of the starting nucleotide peak was measured at each time point and the value was used directly for the calculation. From the rate of peak area reduction or disappearance of the intermediate the rate constant and the half-life of the intermediate calculated as discussed earlier. The result is shown in FIG. 6 which shows that the half-life reduced as much as 30% when just 10 mM Mg2+was used in the cleave mix −26.9 second to 18.3 second.

1. A method for synthesis of DNA or RNA strands comprising the steps:
a) providing a primer capable of binding to nucleotides;
b) providing a first nucleotide according to general formula (I): 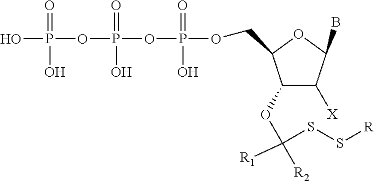
with
B=natural or non-natural nucleobase
R=—Me, —Et, —Pr, —iPr, —iBu, CH₂CH₂NH₂, CH₂CH₂CN
R1, R2=H, D, —Me, —Et, —Pr, —iPr, —iBu, CHF₂ CH₂CH₂F;
c) providing terminal transferase enzyme thereby ligating the first nucleotide to the primer;
d) cleaving and removing the protection group SS-R from the first nucleotide; and
e) repeating steps b) to d) by providing further nucleotides according to general formula
(I) thereby obtaining DNA or RNA strands.

2. The method according to claim 1 characterized in that the primer is a DNA or RNA strand.

3. The method according to claim 1 characterized in that the primer is a DNA or RNA strand bound to a solid surface.

4. The method according to claim 1 characterized in that X is selected from the group consisting of hydrogen, hydroxyl, halogens, —OMe, —OEt, —OCH₂SSMe, —OCH₂CH₂NH₂.

5. The method according to claim 1 characterized in that step c) is performed by providing a cleave reagent.

6. The method according to claim 5 characterized in that the cleave reagent is selected from the group consisting of thiols, dithiolthreitol (DTT), dimercapto propanesulfonic acid (DMPS), phosphines, tris (2-carboxyethyl) phosphine (TCEP), tris (3-hydroxypropyl) phosphine (THPP).

7. The method according to claim 1 characterized in that step c) is performed by providing divalent ions.

8. The method according to claim 1 characterized in that step c) is performed in a pH range of 7-10.

9. The method according to claim 1 characterized in that the first and further nucleotides provided in step b) and e) according to general formula (I) do not comprise a detectable label.
